(19) United States
(12) Patent Application Publication
(10) Pub. No.: US 2011/0034886 A1
(43) Pub. Date: Feb. 10, 2011

(54) IMPLANTABLE MEDICAL DEVICE TOOL AND METHOD OF USE

(75) Inventors: Michael E. Elbe, Fayetteville, GA (US); A. David Smith, Fayetteville, GA (US)

Correspondence Address: ANGIODYNAMICS, INC., 14 PLAZA DRIVE, LATHAM, NY, 12110, US

(73) Assignee: AngioDynamics, Inc., Queensbury, NY (US)

(21) Appl. No.: 12/536,844

(22) Filed: Aug. 6, 2009

(51) Int. Cl. A61B 17/00; A61M 31/00

(52) U.S. Cl. 604/288.01; 606/190

(57) ABSTRACT

A medical device tool for use in combination with an implantable medical device that is capable of being subcutaneously implanted in a patient. The tool has a unitary body having an upper portion and a lower portion and a proximal end and a distal end which has a working section having a leading peripheral edge, an intermediate section having a rounded outer edge, and a handle portion that is positioned at the proximal end of the tool and is capable of being maneuvered. The tool is configured for selective receipt of an implantable medical device.

### FIELD OF THE INVENTION

The present invention relates to a medical device and method, and more particularly, a device, kit, and method for creating a subcutaneous pocket for implantable medical devices, such as an implantable port.

### BACKGROUND

Implantable medical devices are commonly used to temporarily or permanently access a patient's vascular access system. For example, temporary access to a patient's system can be established by percutaneously introducing a needle through the patient's skin and into a blood vessel. This technique is generally suitable for intravenous drug delivery, intravenous feeding, and other short term applications. However, such temporary techniques are not typically suitable for more long term procedures, such as hemofiltration, chemotherapy, hemodialysis, peritoneal dialysis, and other extracorporeal procedures that need to be repeated throughout the lifetime of a patient.

For hemodialysis patient's, one conventional method for long-term vascular access is to surgically create a subcutaneous arteriovenous (A-V) fistula. Conventionally, an A-V fistula is created by anastomosing an artery to a vein, such as, for example, anastomosing the radial artery to the cephalic vein. Once this occurs, the vein becomes dilated and arterializes, thereby becoming suitable for repeated needle punctures. Alternatively, A-V fistulas are also conventionally created by implanting synthetic blood vessels, typically PTFE tubes.

Implantable medical devices such as ports have been used as an alternative to A-V fistulas in such procedures as drug delivery, hemofiltration, hemodialysis, and other treatments. Implantable access ports typically have a reservoir inside of the port, a septum, an outlet, and in some cases, a stem to which a catheter can be attached. The septum of the port is typically a needle-penetrable septum which permits the percutaneous penetration of a needle into the internal reservoir of the device, which is in fluid communication with the catheter and hence to a blood vessel. If the catheter is attached to a vein, it is typically an indwelling catheter.

The use of implantable access ports in the art of drug therapy is well known. In this technique an access port is implanted beneath the subcutaneous layers of a patient's skin. The use of these implantable access devices reduces the trauma otherwise associated with multiple punctures of the skin, or the inconvenience of an externalized catheter for patient treatment purposes. For example, implantable access ports are conventionally used to facilitate frequent blood sampling or to provide for the delivery of medications, nutritions, blood products, and imaging solutions into the patient's blood stream, or to a desired treatment site within the patient. Access to the implanted access port is typically accomplished by percutaneous needle insertion through the patient's skin into the access port through the penetrable septum or other similar structure by using a non-coring hypodermic needle.

Conventionally, access ports are surgically and/or radiologically placed. A surgical cutdown procedure is the most common method used to create a pocket in the patient's skin for the placement of the access port. During this procedure, the implantable access port is typically positioned in the anterior chest wall using ultrasound while a patient is under intradermal and subcutaneous anesthesia. Before the implantable port is placed in the chest wall, a suitable site for the port is chosen in the upper chest wall, a few centimeters below the clavicle. Before a subcutaneous pocket is created by the surgeon, a firm, bony area on the patient is conventionally selected, which enables the access port to be supported during operational access.

After the site is chosen, the surgeon prepares the subcutaneous pocket within the patient's skin in which to place the implantable port. The surgeon typically uses a scalpel to create a generally vertical incision within the patient's skin that is 2-3 cm deep just below the site chosen for the access port in the skin and then extends the incision laterally so that the access port can be placed in the pocket that is offset from the vertical incision, caudal to the clavicle and laterally offset from the main portion of the tissue pocket so that the access port can be disposed under skin which has not been surgically penetrated. This allows a needle or other access device which is percutaneously penetrated into the access port to avoid passing through the vertical incision and also avoids having the skin incision site overlie the port access dome or septum. The port pocket is generally created underneath the skin such that an incision is not be made directly on top of the implanted port, but is preferably to either side of it.

Conventionally, the port pocket is created deeply enough below the skin to allow the port to be implanted without invoking potential erosion issues, which can happen if the port is too shallowly placed. In one example, an overlying tissue thickness of approximately 0.5-2 cm is used. If the incision is made directly over the port, this could also cause skin erosion and infection. In some cases, a rod, tube, or an access cannula can be used for subsequent access to the port, and is left in place, remaining anchored in the aperture for a time sufficient to create the access tract before the port is placed in the pocket.

Subsequently, blunt dissection is used to create and enlarge the pocket to accommodate the size of the implantable access port superior to the skin incision so that the access port can be easily inserted into the subcutaneous tissues. In some cases, conventional dissection tools such as scalpel, which can have a sharp or serrated cutting edge configured for dissecting tissue, can be used. Alternatively, some surgeons or physicians can use their thumb or finger to create a port pocket having the desired shape and size. The blunt dissection can cause discomfort as it is known that the use of sharp and/or serrated dissection tools or the use of a doctor's hand can be painful to the patient and can cause trauma to the skin and surrounding tissue. In one aspect, the size of the port pocket is important. Typically, the surgeon uses his or her best judgment in determining the size of the pocket needed to accommodate a port. In one preferred conventional surgical technique, the pocket is formed with a dimension sufficient to accommodate the access port while also allowing easy closure of the incision.

After the port pocket is created, the access port is placed in the pocket with the septum positioned such that it underlies the patient's skin at the desired depth. A tunnel is then made from the puncture site to the pocket with the tunneling device, and the catheter is pulled through the tunnel. The catheter tubing is tunneled to the venipuncture site and connected to the port. The port catheter is then typically trimmed to a desired length. The catheter tubing can be connected to the port before or after the port is inserted into the port pocket. Then the catheter is flushed. After insertion of the port into the port pocket of the patient's skin, the position of the catheter tip is determined using ultrasound. The port can then be sutured to the patient's fascia to prevent port rotation. The incision is then closed up, and the puncture site is stitched up using subcutaneous sutures. If the port pocket is the appropriately sized, the need to suture the port to the deep fascia to prevent port migration or rotation can be minimized or eliminated. In operation, the implanted port can then be accessed conventionally, such as, for example, using a Huber needle.

There are several potential complications that could occur when ports are implanted in the patient's skin using the implantation procedures described above. If a sharp dissection instrument is used, this could cause trauma or even injury to the patient. In other instances, the pocket that is initially created by a practitioner could either be too large or too small. If the port pocket size is too large, the patient could be compromised and excessive tissue loss could occur. Additionally, the patient might need additional suturing to the fascia in order to close the pocket. Large tissue pockets can also increase the likelihood of hematoma and infection. Conversely, if the formed tissue pocket is too small, the surgeon can have to take additional time to enlarge the pocket prior to implanting the port. Either of these problems can lengthen procedure time, cause additional trauma to the patient, and provide an increased risk of infection. Other complications can include bacteremia, hematoma, skin necrosis, port rotation, seroma, and other various types of infections, any of which can require the port to be removed and/or replaced with a new implantable port.

What is needed is a dissection tool that allows a practitioner to create an appropriately sized and shaped port pocket such that the patient's health is not compromised, excessive tissue loss does not occur, and the port itself is not compromised. What is presented herein is a medical device implantation tool, kit, and method involving minimal steps that can be used for to create pockets within a patient's tissue in order to allow implantable medical devices of various sizes to be implanted within a patient's skin. The device and method provided herein simplifies the procedure for creating tissue pockets in patients for the implantation of medical devices and allows a practitioner to create custom-sized tissue pockets that are optimized for the size of the implantable medical device.

Various other purposes and embodiments of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention can be found in the Detailed Description.

### SUMMARY

A medical device tool is provided herein for use in combination with an implantable medical device that is capable of being subcutaneously implanted in a patient. In one aspect, the tool has a unitary body having an upper portion and a lower portion and a proximal end and a distal end which has a working section having a leading peripheral edge, an intermediate section having a rounded outer edge, and a handle portion that is capable of being grasped and selectively maneuvered. In one aspect, the handle is connected to the working section and extends longitudinally to the proximal end of the tool. In an additional aspect, the tool can be configured for selective receipt of an implantable medical device.

These and various other objects, advantages and features of the invention will become apparent from the following description and claims, when considered in conjunction with the appended drawings. The invention will be explained in greater detail below with reference to the attached drawings several embodiments of the present invention in which the same reference characters denote similar elements.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A illustrates a perspective view of a medical device tool.

FIG. 1B illustrates a bottom perspective view of the medical device tool of FIG. 1A.

FIG. 2A illustrates a top view of the medical device tool of FIGS. 1A and 1B.

FIG. 2B illustrates a bottom view of the medical device tool of FIG. 2A.

FIG. 3 illustrates a side view of the medical device tool of FIGS. 2A and 2B.

FIG. 4A illustrates a front end view of the medical device tool of FIG. 3.

FIG. 4B illustrates a back end view of the medical device tool of FIG. 3.

FIG. 5 illustrates a perspective view of another embodiment of a medical device tool.

FIG. 6A illustrates a top view of the medical device tool of FIG. 5.

FIG. 6B illustrates a cross-sectional view of the medical device tool of FIG. 5, taken along line 6B of FIG. 6A.

FIG. 7A illustrates the medical device tool of FIG. 1 being inserted into the skin during a method of using the medical device tool.

FIG. 7B illustrates the medical device tool being used to selectively dissect and urge or push tissue away.

FIG. 7C illustrates the medical device tool being used to surgically form a subcutaneous tissue pocket having a desired size and shape.

FIG. 7D illustrates the medical device tool being used to position an implantable medical device that is selectively positioned therein a cavity defined in the tool therein the formed subcutaneous tissue pocket.

FIG. 7E illustrates the implantable medical device after it has been subcutaneously implanted within the skin.

FIG. 8A illustrates a perspective view of the medical device tool in relation to an implantable medical device that is selectively positioned therein a cavity defined in a proximal portion of the tool.

FIG. 8B illustrates a bottom perspective view of the medical device tool and implantable medical device of FIG. 8A.

FIG. 8C illustrates a cross-sectional view of the medical device tool, showing a side view of the implantable medical device selectively positioned therein the cavity defined in the proximal portion of the tool, taken along line 8C of FIG. 8A.

### DETAILED DESCRIPTION OF THE INVENTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or can not occur, and that the description includes instances where said event or circumstance occurs and instances where it does not. As is conventional, the term “distal” means away from the physician when the catheter is being or has already been inserted into a patient, while the term “proximal” means closest to or toward the physician when the catheter is being or has already been inserted into a patient. The dimensions provided herein are for exemplary purposes only, and the scope and contents of the present disclosure, particularly the claims, should not be limited to such dimensions.

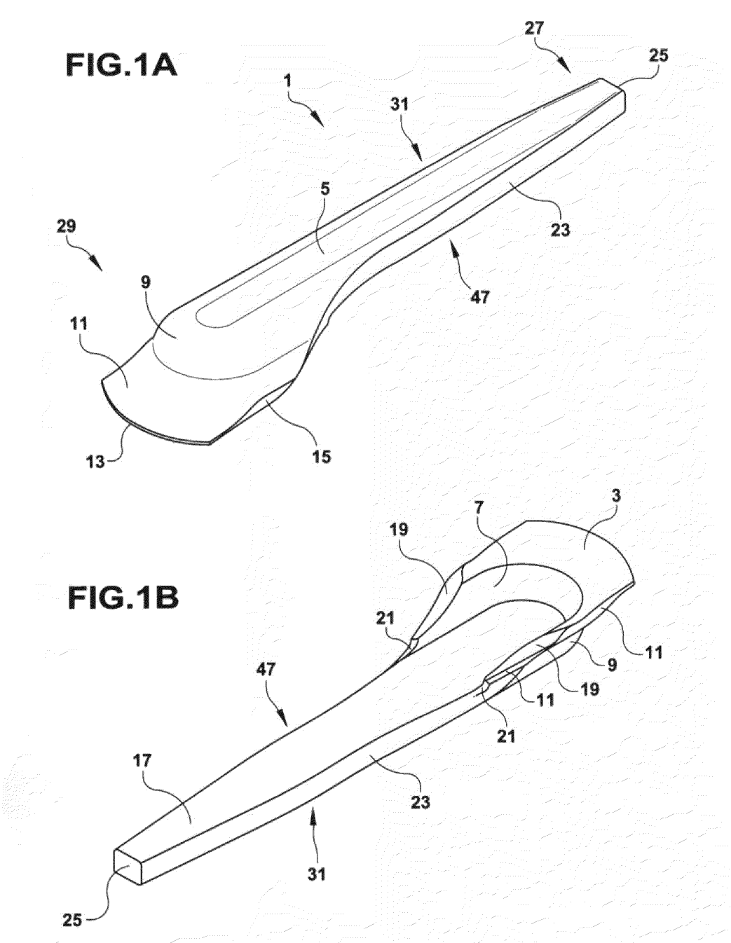

Referring now in detail to the drawings, in which like reference numerals indicate like parts or elements throughout the several views, in various embodiments, and referring to FIGS. 1-8C, presented herein is an exemplary implantable medical device pocket creation tool, a kit, and a method of using the device to create a custom-size port pocket and to subcutaneously insert an implantable medical device into a patient.

FIGS. 1A through 4B illustrate one embodiment of a medical device tool. As illustrated in FIG. 1A, the medical device tool 1 has a top portion 31 and a bottom portion 47 and a proximal end 27 and a distal end 29. In one aspect, at least one side surface 23 is positioned between the proximal end 27 and the distal end 29 of the tool 1. In one aspect, the side surface 23 extends substantially horizontally along a length of the tool 1 from the proximal most edge of the working section 11 toward the proximal end 27 of the tool 1.

In one aspect, the tool 1 also comprises a working section 11, a peripheral edge 13, at least one side edge 15, at least one side surface 23, and a back surface 25. In one aspect, the working section 11 has a proximal end and a distal end. In one aspect, the working section 11 can be biocompatible such that it can be inserted into a port pocket in a patient's skin. The proximal end of the working section 11 can be flush with at least a portion of the intermediate section 9. In another aspect, the working section 11 can be contiguous with and extend distally of the intermediate section 9. The working section 11 can be comprised of an outer surface, a forward facing peripheral edge 13, and at least one side edge 15. In one aspect, the outer surface of the working section 11 can be positioned at an obtuse angle relative to a working section plane that bisects the peripheral edge 13. In one non-limiting example, the obtuse angle can be between about 100° to 130°, preferably between about 110° to 120°, and more preferably about 116°. In one aspect, each side edge 15 of the working section 11 can be positioned on either side of the working section 11 and can extend substantially transverse to the working section plane. In another aspect, at least a portion of the working section 11 can have a sloping or slightly concave outer surface. In one non-limiting example, the radius of curvature can be between about 0.025 to 0.045, preferably between about 0.030 to 0.040, and more preferably about 0.350. In one aspect, at least a portion of the working section 11 can comprise radiopaque markers, depth markers, or other types of indicia, as desired, to assist a practitioner in placing the tool 1 within the patient.

In one aspect, the tool 1 comprises a top section 5, an intermediate section 9 with a front rounded edge and longitudinally extending edges. In one aspect, the intermediate section can be substantially U-shaped. In a further aspect, it is contemplated that the longitudinally extending edges of the intermediate section can gradually taper or become narrower from the distal end 29 of the tool 1 toward the proximal end 27 of the tool 1. In one aspect, the intermediate section 9 can be substantially uniform in width. In one aspect, the intermediate section can be between about 0.250 to 0.280 inches, preferably between about 0.255 to 0.275 inches, and more preferably about 0.262 inches in width. In another aspect, the top portion 5 can extend along the longitudinal length of the tool 1 between about 3.500 to 4.200 inches, preferably between about 3.600 to 4.100 inches, and more preferably about 3.881 inches in length. In another aspect, the intermediate section 9 can extend substantially therebetween the front rounded edge of the intermediate section 9 toward the proximal end of the tool 1.

In one aspect, portions of the longitudinally extending edges of the intermediate section 9 are positioned between the top surface 5 and the at least one side surface 23. In one exemplary embodiment, and as illustrated in FIG. 1A, the top section 5 is positioned in a center region of the top portion 31 of the tool 1 and is flush with and contiguous with at least a portion of the intermediate section 9. In one aspect, the outer rounded portion of the intermediate section 9 of the tool 1 can have an outer radius of curvature that is a blunt, non-traumatic traumatic leading edge that is designed to be conducive for blunt dissection of the tissue to create a tissue pocket. In one aspect, the outer rounded portion of the intermediate section 9 can have a convex outer surface with a radius of curvature between about 0.270 to 0.310, preferably between about 0.280 to 0.300, and more preferably about 0.291.

Referring now to FIG. 1B, in one aspect, the intermediate section 9 of the tool 1 comprises an inside surface 17 that is spaced from and opposes the top section 5. In one aspect, the inside surface 17 can be substantially parallel to the top section 5. In another aspect, the bottom portion 47 of the tool can comprise an inner surface 7. In one aspect, the inner surface 7 can positioned substantially transverse to the inside surface 17. However, it is contemplated that the transition between the inner surface and the inside surface 17 can be curved, i.e., for example and without limitation, the radius of curvature between the inner wall 7 and the inside surface 17 can be about 0.270 to 0.310, preferably between about 0.280 to 0.300, and more preferably about 0.290.

In one aspect, the inside surface 17 of the tool can be positioned between the respective side surfaces 23. In one aspect, the working section 11 comprises a bottom surface 3 that is adjacent to and contiguous with at least a portion of the inner surface 7 of the intermediate section 9. In one aspect, the inner surface 3 and the inner surface 7 can be positioned substantially orthogonally in relation to one another. In one aspect, the radius of curvature of the junction between the inner surface 3 and the inner surface 7 is about 0.375 to 0.416, preferably between about 0.385 to 0.406, and more preferably about 0.396. Optionally, it is contemplated that at least a portion of the inner surface 3 is curved. It is contemplated that an inner surface 3 having a substantially convex shape can aid the practitioner during blunt dissections by helping to smooth tissue when a port pocket is being created and dissected.

In one aspect, the back edge 25 of the tool 1 can be in the shape of a square or a rectangle, although any suitable shape can be used for the back edge 25 of the tool. In one exemplary aspect, the back surface 25 of the tool 1 can have a width of about 0.225 to 0.275 inches, preferably between about 0.235 to 0.265 inches, and more preferably about 0.250 inches. In one exemplary aspect, and without limitation, the port pocket tool 1 can be approximately 4.5 inches in length, approximately 0.5 inches in height at the distal end 29 of the tool 1, and approximately 0.189 inches in height at the proximal end 27 of the tool 1. In

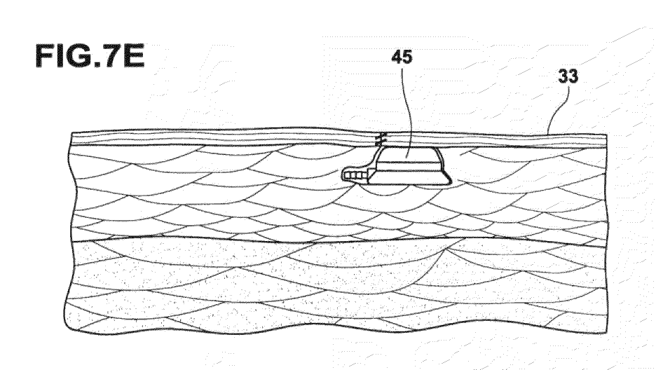

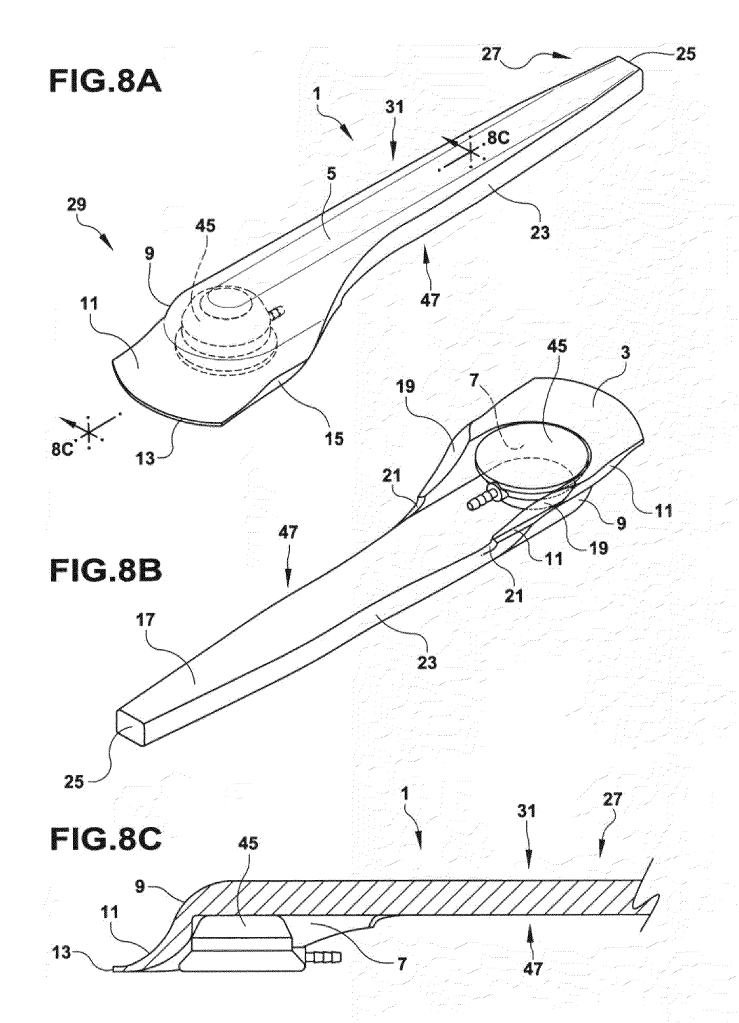

In one aspect, and as illustrated in FIG. 1B, a cavity can be defined by the inner surface 7 and the inside surface 17 of the port pocket creation tool 1. In one aspect, the cavity defined by the inner surface 7 and the inside surface 17 of the tool 1 can be sized and shaped to form a releasable interference fit with outer surface of an implantable medical device 45, as further illustrated in FIG. 7D and FIGS. 8A-8C. Of course, it is also contemplated that the cavity can be sized and shaped for the operative receipt of a desired implantable medical device 45. As one skilled in the art will appreciate, the cavity formed in the tool 1 can allow the desired implantable medical device, such as, for example and without limitation, an implantable medical port, to be inserted into and selectively released therefrom, the cavity. In this aspect, the size of the formed pocket can be optimized for the desired shape and size of implantable medical devices.

In one aspect, respective portions of the top section 5, the intermediate section 9, side sections 23, inside surface 17, and the back surface 25 can comprise a handle portion of the tool 1. The port pocket creation tool 1 can be manufactured such that the dimensions of the tool are conducive for gripping by a practitioner's hand during use of the tool to create tissue pockets for implantable medical devices. In another aspect, the tool 1 can be manufactured using a clear plastic material which can allow for better visualization of the surgical field and the tool 1 while dissection is being performed and/or to better view the port while the port 45 is being placed within the port pocket. In one aspect, the handle can be designed for being grabbed in order to manipulate the tool 1 during tissue dissection in order to better control tissue dissection while creating a port pocket. In one aspect, it is contemplated that the handle can be contoured or rounded instead of having right angles or sharp cornered angles. In a further aspect, the respective contours of the handle can be configured such that the handle is adapted to fit various sizes of hands. In another aspect, at least a portion of the handle can comprise ribs to facilitate gripping by a human hand.

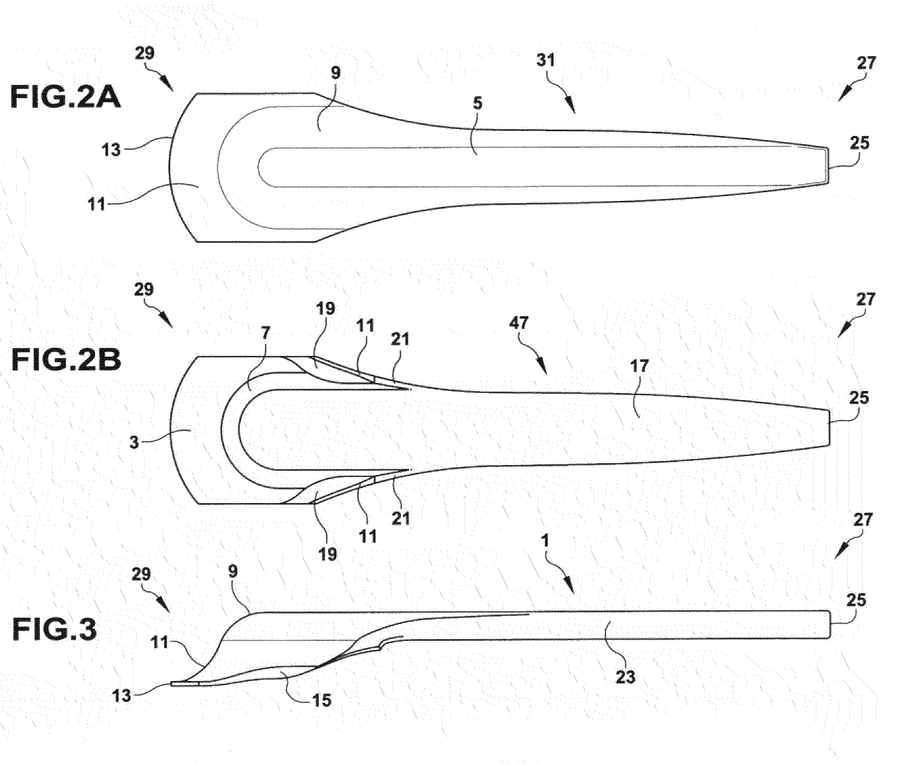

In one exemplary aspect, and as shown in FIG. 2A, the peripheral edge 13 can be rounded or semi-circular so as to allow a practitioner to more easily push away tissue. In another aspect, as illustrated in FIG. 2A, the front peripheral edge 13 of the tool 1 can have a curved or radiused edge that allows the practitioner to be better able to perform non-traumatic blunt tissue dissections. In one aspect and without limitation, the peripheral edge 13 of the working section 11 can have a height of approximately 0.025 inches and an arc length of approximately 1.095 inches. In another aspect, the radius of curvature of the peripheral edge 13 can be between about 0.600 to 0.900, preferably between about 0.675 to 0.825, and more preferably about 0.750. In one aspect, the semi-circular shape of the working section 11 can be sized such that it is larger in size compared to the size of an implantable medical device 45. As illustrated in FIG. 2A, in one aspect, although the corner edges of the front peripheral edge 13 can be pointed or slightly sharpened, in an alternative aspect, it is contemplated that the edges can be rounded, so as to decrease any trauma to the patient.

In one aspect, each of the contoured surfaces 19 can be positioned on either side of the inner surface 7 such that each contoured surface 19 substantially tapers outwardly from the inner surface 7 toward the outer side edges of the tool 1. In one aspect, each of the contoured surfaces 19 can be contiguous at their distal most edges with the respective proximal edges of the inner surface 3. In one aspect, the port pocket creation tool 1 can also comprises at least one inner edge surface 21. In one aspect, a distal most edge of each of the inner ledge surfaces 21 can be contiguous with a proximal most edge of each of the respective contoured surfaces 19. In one aspect, a proximal most portion of each of the inner ledge surfaces 21 can transition into each of the respective side surfaces 23. As illustrated, contoured surfaces 19 are positioned on each side of the inner surface 7. In one aspect, each of the contoured surfaces can be substantially tapered to minimize trauma during blunt dissections. In one aspect, inner surfaces 21 can comprise a blunt, non-sharpened, non-traumatic leading edge to further minimize trauma during blunt dissections.

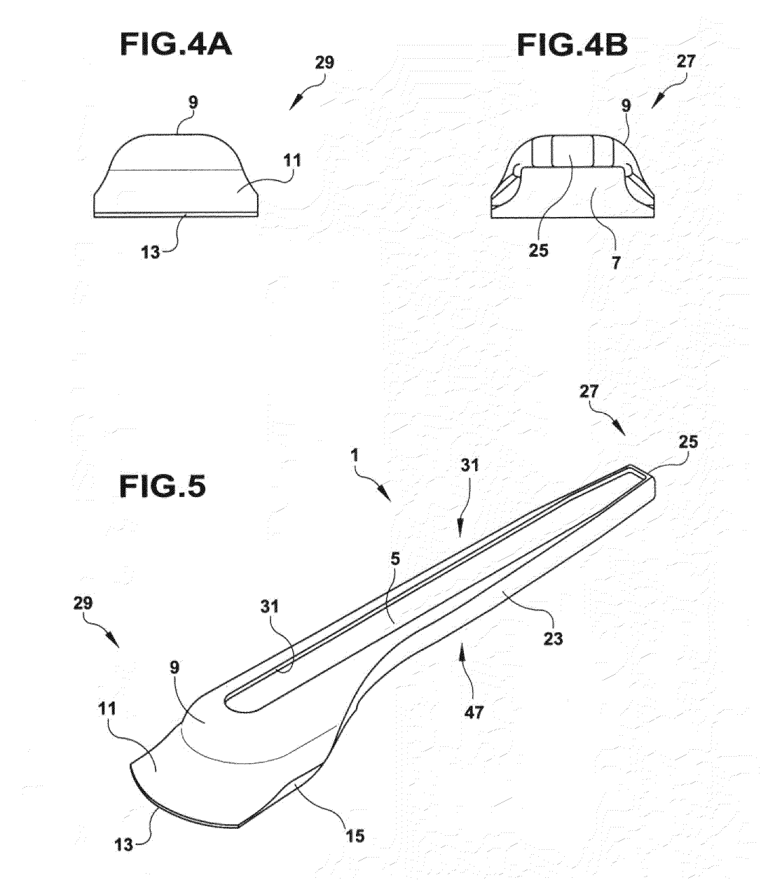

Referring to FIG. 3, a side view of the tool 1 is provided. In this aspect, the distal portion of the tool 1 has a rounded outer surface 9 with a radius of curvature that allows a practitioner to create a pocket having a desired size and shape for insertion of an implantable medical device. In one aspect, and without limitation, the total height of the distal portion 29 can be approximately 0.5 inches, while the height of the proximal portion 27 is approximately 0.189 inches.

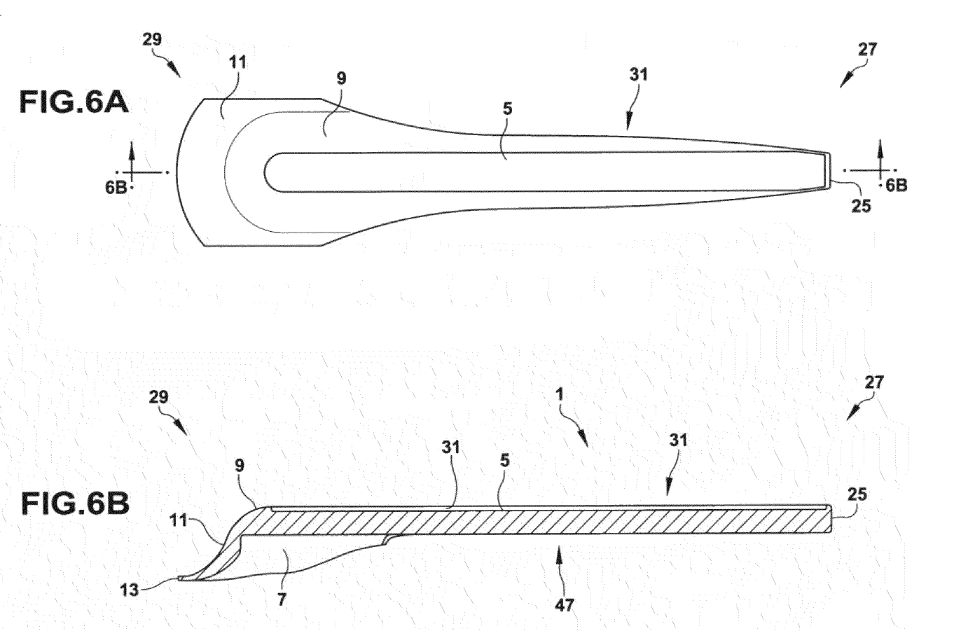

Referring to FIGS. 4A and 4B, as viewed from the distal end of the device 1, the peripheral edge 13 can be joined to and contiguous with the working section 11 at one end of the working section 11, and the intermediate section 9 is joined to and contiguous with working section 11 at the other end of the working section 11. In another exemplary aspect and without limitation, the tool 1 can have a width at the proximal end 29 of the tool 1 along the peripheral edge 13 of the working section 11 of approximately 1.0 inch. In one aspect, the tool 1 can provide an overall width profile that is non-traumatic and is suitable for blunt dissections within a patient's tissue. In another aspect, and as illustrated in FIG. 4B, it is contemplated that the proximal most end 25 of the tool 1 is smaller in width compared to the distal end 29 of the tool 1.

As described above, and as illustrated in FIG. 4B, the inner surface 7 can define a cavity that is sized and shaped for the operative receipt of the desired implantable medical device. It is contemplated that the inner surface can have a shape that mirrors or mimics the outer surface of the implantable medical device. In one aspect, the implantable medical device can be a port that is configured to be coupled to a catheter. In another aspect, the overall shape of the distal end of the port tool 1 can be configured to have any shape that is suitable for the outer profile of an implantable medical device. In one non-limiting example, the distal end 29 of the tool 1 can have an elliptical, oval, rectangular, or square shape, or any suitable shape thereof.

In one aspect, the tool 1 can be manufactured from any suitable plastic material using a molding process. For example, in one aspect, the tool 1 can be made of any suitable plastic material, such as, but not limited to, polyurethane, elastomers, and the like. In one exemplary aspect, the tool 1 can be made as unitary body. In one aspect, the tool 1 can be a one piece device that is used to subcutaneously create a tissue pocket underneath a patient's skin in order to enable a practitioner to insert implantable medical devices such as implantable ports into a patient's skin. In another aspect, the tool 1 can have a non-traumatic dissection edge that is an integral part of the tool 1 itself. In yet another aspect, the working section 11 can be detached from the distal portion 29 of the tool 1. In yet another aspect, the handle portion of the device can be disposable. In one aspect, the tool 1 can be manufactured for all model types and sizes for which it is deemed applicable. In one exemplary aspect, multiple versions of the tool 1 can be manufactured in order to optimize the pocket size that can be created for different types and sizes of medical devices. Although in one aspect the tool 1 can be designed to be a single-use, disposable instrument that is discarded after use. Optionally, the tool 1 can be used repeatedly and can be sterilized between uses.

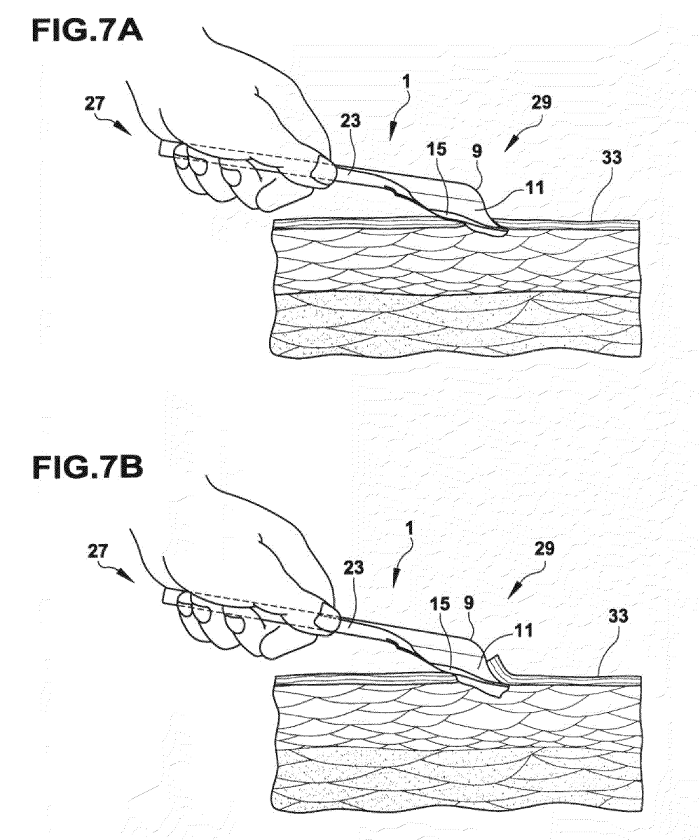

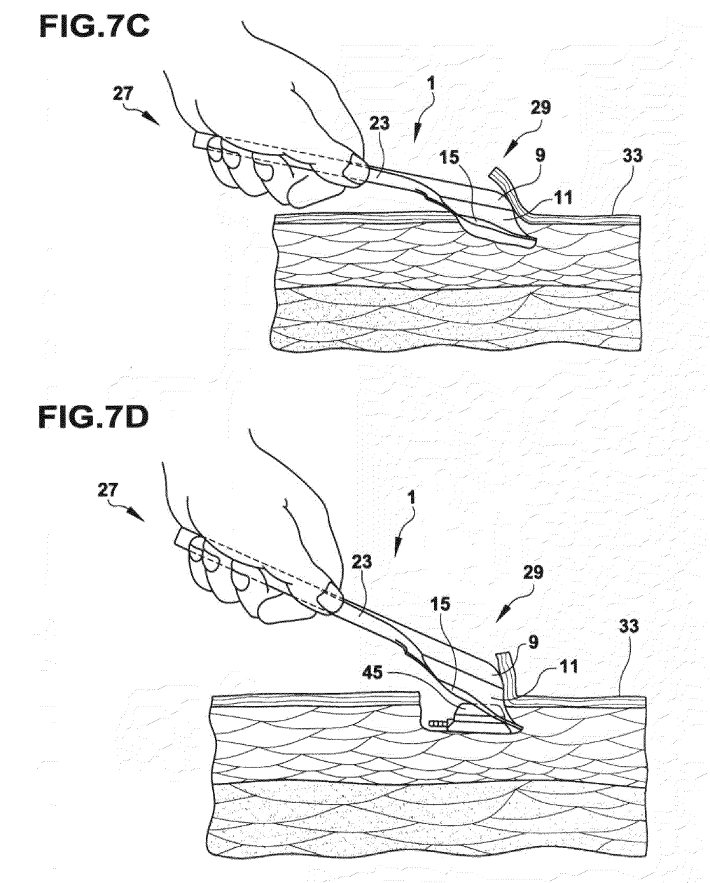

FIGS. 5 through 6B illustrate a second embodiment of the medical device tool 1. In this aspect, the tool 1 has a top section 5 that is recessed. In one aspect, the recessed portion 5 has an inner edge or wall 31 that helps to define the recessed portion. This recessed portion 5 allows a practitioner to better able to be able to grip and manipulate the handle portion of the tool 1. In one aspect, as illustrated in FIG. 6B, the inner ledge 31 can have a height of approximately 0.025 inches.

FIGS. 7A through 8C illustrate a method of using the medical device tool. While grasping the handle portion of the tool 1 as described above, a surgeon can manipulate the tapered working section 11 to separate the underlying tissue, thereby creating a pocket for the port. Once the desired port pocket is created, the surgeon can then use the tool 1 to push upper tissue upward and to place an implantable medical device, such as, but not limited to, a port, into the newly created pocket between the working section 11 of the tool 1 and lower tissue layers.

In one aspect, the cavity that is defined within the port pocket creation tool 1 can be positioned to allow an implantable medical device such as a port 45 to slide into the pocket after it is created, while a practitioner uses the tool 1 to hold back tissue and keep the pocket area open. In one aspect, the tool 1 can then be withdrawn, leaving the port 45 in the pocket, as illustrated in FIG. 7E. Typically, before the implantable medical device is inserted into the patient's skin, a substantially vertical incision of a desired depth is created in the patient's skin to allow for the deposit of an implantable medical port at a desired depth. The leading peripheral edge of the tool 1 is advanced through the generally vertical incision in the patient's tissue, the handle of the tool is then manipulated such that the leading peripheral edge of the tool lies substantially horizontally, and the leading peripheral edge is advanced horizontally to create the subcutaneous pocket. The horizontal advancement of the leading peripheral edge of the tool results in the creation of a port pocket that has a substantially semi-circular outer geometrical profile to mimic the outer profile size of the implantable medical device.

In this method described herein, a dissection tool having a blunt, non-traumatic leading edge is provided, as illustrated in FIG. 7A. The blunt, non-traumatic leading edge of the tool is then advanced through the patient's tissue to create a subcutaneous pocket, as illustrated in FIGS. 7B and 7C. As illustrated in FIG. 7D, an implantable medical device 45 having an outer perimeter substantially corresponding to an outer geometrical profile of the blunt, non-traumatic leading peripheral edge of the tool is then implanted within the subcutaneous pocket.

In one aspect, the tool 1 described herein allows a practitioner to create a pocket under the skin that is optimally sized for the implantable medical device such as a port 45. In one aspect, this tool 1 can enable the user to reduce any unnecessary cutting of the patient's tissue and can reduce any excess trauma to the area where the pocket is created. Thus, the tool 1 described herein provides an alternative for practitioners who currently do not have a dedicated instrument for the creation of a pocket in which to place the port and who often resort to using whatever is at hand, such as scalpels, forceps, and even their fingers. The tool 1 provides surgeons a dedicated instrument with which to make an optimal, custom-sized tissue pocket for an implantable medical device, such as, but not limited to, an implantable medical port. This tool 1 allows physicians to avoid the need to create pockets manually, such as with their fingers, or with sharp or traumatic tools that might be uncomfortable or harmful to the patient, and it allows the physician to create a port pocket that is tailored to the size of the port, which allows for a more snug fit between the port and the port pocket creation tool. This also decreases the chance that the port will flip over within the port pocket.

In one aspect of this invention, a kit is provided. The kit can comprise a tool 1 having a working section 11 that can have different shapes or sizes, as described above, and a container to hold the implantable medical device. In one aspect, the container can be a bag, a box, tray, or other such storage device. In one exemplary aspect, the kit can also comprise an implantable medical device, such as, but not limited to an implantable medical port 45. The dissection tool 1 can be sterilized before it is placed in the container. Instructions for use (IFU) can also be provided for a practitioner. The IFU can pertain to a method of using the dissection tool or implanting the dissection tool within the patient and inserting an implantable medical device into a previously created port pocket that was created by a patient. It is contemplated that the instructions for use can describe methodologies for using the implantable medical device tool to create a subcutaneous tissue pocket. In one aspect, the tool 1 can be packaged in the port kit with the other kit components. Thus, in one aspect, it is contemplated that a kit cab be provided that comprises an implantable medical device, an implantable medical device dissection tool having an outer leading peripheral edge corresponding to the outer geometrical profile of the implantable medical device, and a container capable of containing the implantable medical device and the implantable medical dissection tool. Packaging can also be provided to suitably surround the implantable medical device and the implantable medical device dissection tool.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. The words “including” and “having,” as used herein including the claims, shall have the same meaning as the word “comprising.” Those familiar with the art can recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the selected embodiments of the invention. Those skilled in the art can recognize other equivalents to the specific embodiments described herein which equivalents are intended to be encompassed by the claims attached hereto.

What is claimed is:

1. A medical device tool for use in combination with an implantable medical device to be subcutaneously implanted in a patient, comprising:
a unitary body having an upper portion and a lower portion and a proximal end and a distal end, wherein the distal end of the unitary body comprises a working section having a leading peripheral edge, wherein the unitary body further comprises:
an intermediate section connected to the working section, the intermediate section having a rounded outer edge; and
a handle portion formed from at least a portion of the intermediate section, wherein the handle extends longitudinally to the proximal end of the tool,
wherein the tool is configured for selective receipt of the implantable medical device.

2. The medical device tool of claim 1, wherein at least a portion of the leading peripheral edge comprises a semi-circular blunt leading peripheral edge.

3. The medical device tool of claim 1, wherein the working section has a concave outer surface and a convex inner surface, wherein the convex inner surface and the concave outer surface are in space opposition to one another.

4. The medical device tool of claim 1, wherein a cavity is defined in the lower portion of the unitary body, and wherein the cavity is configured for selective receipt of the implantable medical device.

5. The medical device tool of claim 4, wherein the cavity is configured to form interference fit with the implantable medical device upon selective receipt of the implantable medical device.

6. The medical device tool of claim 1, wherein the implantable medical device is an implantable port.

7. A method for subcutaneously implanting a medical device in a patient, wherein the method comprises:
providing a dissection tool having a blunt, non-traumatic leading edge,
advancing the blunt, non-traumatic leading edge of the tool through a patient's tissue to form a subcutaneous pocket within the patient having an outer perimeter substantially corresponding to an outer geometrical profile of the blunt, non-traumatic leading peripheral edge of the tool; and
implanting the implantable medical device within the formed subcutaneous pocket.

8. The method of claim 7, further comprising
creating an initial incision in the patient's skin to a desired depth beneath the patient's skin,
advancing the leading peripheral edge of the tool through the generally vertical incision,
manipulating the handle of the tool such that the leading peripheral edge of the tool lies substantially horizontally, and
advancing the leading peripheral edge horizontally to form the subcutaneous pocket.

9. The method of claim 8, wherein advancing the leading peripheral edge of the tool horizontally forms a port pocket that has a substantially semi-circular outer geometrical profile.

10. A kit comprising:
an implantable medical device,
an implantable medical device dissection tool comprising:
a unitary body having an upper portion and a lower portion and a proximal end and a distal end, wherein the distal end of the unitary body comprises a working section having a leading peripheral edge, wherein the unitary body further comprises:
an intermediate section connected to the working section, the intermediate section having a rounded outer edge; and
a handle portion formed from at least a portion of the intermediate section, wherein the handle extends longitudinally to the proximal end of the tool,
wherein the implantable medical device dissection tool has an outer leading peripheral edge corresponding to the outer geometrical profile of the implantable medical device, and
a container capable of containing the implantable medical device and the implantable medical device dissection tool.

11. The kit of claim 10, further comprising packaging configured to operationally surround the implantable medical device and the implantable medical device dissection tool.

12. The kit of claim 11, further comprising instructions for use, wherein the instructions for use describe methodologies of using the implantable medical device tool to form a subcutaneous tissue pocket.
